(19) United States
(12) Patent Application Publication
(10) Pub. No.: US 2026/0272683 A1
(43) Pub. Date: Sep. 17, 2026

(54) BILIARY STENT AND METHOD MANUFACTURING THEREOF

(71) Applicant: MERIL HEALTHCARE PVT. LTD, Vapi (IN)

(72) Inventors: PRAMOD KUMAR MINOCHA, Vapi (IN); DEVESHKUMAR MAHENDRALAL KOTHWALA, Surat (IN); ARPIT PRADIPKUMAR DAVE, Silvassa (IN); MAHESH LAXMAN PAWAR, Nashik (IN)

(73) Assignee: MERIL HEALTHCARE PVT. LTD, Vapi (IN)

(21) Appl. No.: 19/470,461

(22) PCT Filed: Feb. 16, 2024
(86) PCT No.: PCT/IN2024/050163

§ 371 Date: Sep. 29, 2025

(30) Foreign Application Priority Data
Feb. 2, 2022 (IN) 202221005670
Mar. 30, 2023 (IN) 202323024049

(51) Int. Cl. A61F 2/90

(52) U.S. Cl. CPC A61F 2/90; A61F 2240/001

(57) ABSTRACT

The present invention discloses a method of manufacturing a stent from a biodegradable polymer. The method commences by braiding at least a plurality of first monofilaments and a plurality of second monofilaments to form a braided structure. The braided structure is subjected to a primary shape setting at a predefined temperature for a predefined time duration. Thereafter, at least one of the first monofilaments or the second monofilaments are back braided. The stent is axially compressed to decrease the length (L3) of the stent, and increase the braiding angles of the stent. The stent is subjected to secondary shape setting at the predefined temperature for the predefined time period. The present invention further discloses a stent.

### FIELD OF INVENTION

The present invention relates to a method of manufacturing stents. More specifically, it relates to a method of manufacturing biliary stents.

### BACKGROUND OF INVENTION

Bile ducts are a series of thin tubes extended from the liver to the small intestine. The bile duct carries bile from the liver and gallbladder to the small intestine. Bile is a substance that helps in the digestion of fats. The bile duct stricture is a condition in which the bile duct gets smaller or narrower. The aforesaid condition of the bile duct results in reduction of passing of the bile from liver to the small intestine leading to difficulty in digestion of food. The stricture causes abnormalities in the blood and/or a rise in non-essential liver enzymes. Patients with mild biliary strictures may not show any symptoms. However, when the stricture becomes more pronounced, symptoms start to develop. The symptoms may include abdominal pain on the right side of the body, itching, yellow skin or eyes, fever, nausea, vomiting, gray-colored or pale-colored stool, etc.

The bile duct stricture may be caused due to injury to the bile ducts during surgery, for example, surgery to remove gallbladder, damage, and/or scarring due to gallstone in the bile duct, cancer of the bile duct, liver or pancreas, etc.

Endoscopy is a widely used approach for the treatment of biliary strictures. Endoscopic treatment is performed by implantation of a polymeric stent, followed by further sessions of stenting with multiple polymeric stents. Temporary implantation of multiple polymeric stents has been used in patients having benign biliary strictures. However, the polymeric stents are less than ideal as they are prone to occlusion due to bacterial colonization and/or build-up of biofilm on the surface. Further, polymeric stents are also known to migrate from their implanted position. Moreover, due to their relatively rigid structure, their implantation in an artery of smaller diameter and/or subsequent expansion to fit against artery's walls is difficult to perform.

Self-expandable metal stents are also utilized to treat biliary structures. However, the metal stents are known to be occluded due to tumor in-growth stent interstices. Moreover, the metal stents are permanent implants, therefore, are needed to be removed from the body by surgery, which may cause further complications to the patient. Further, because the bile duct is narrow and/or the stent is to be implanted at a slope, the stent might be at a higher risk of migration after implantation.

The use of biodegradable stents has been used to address the aforesaid problems of stenting in the bile duct. However, conventional bioresorbable stents are prone to the risk of migration due to weak radial forces. Moreover, bioresorbable stents have shown insufficient strength and/or low recovery, leading to elastic recoil of the stent. Further, the bioresorbable stent may not be well adapted to the biliary lumen, thus leading to complications such as inflammation and/or restenosis.

Therefore, there exists a need for an improved stent system that can overcome the limitations of the existing ones.

### SUMMARY OF INVENTION

The present invention relates to a method of manufacturing a stent from a biodegradable polymer. The method commences by braiding at least a plurality of first monofilaments and a plurality of second monofilaments from a first end to a second end at a first predefined braiding angle to form a braided structure. The braided structure defines a length between the first end and the second end. The second end being axially opposite to the first end. The braided structure is subjected to primary shape setting at a predefined temperature for a predefined time duration. At least one of the first monofilaments or the second monofilaments are back braided from the second end to the first end at a second pre-defined braiding angle to form the stent. The stent is axially compressed by applying a pre-defined force to decrease the length by 25% to 10% of the length of the stent, and increase the first and second predefined braiding angle of the stent by 25% to 6.25% of the first and second pre-defined angle of the stent. The stent is subjected to a secondary shape setting at the predefined temperature for the predefined time period.

The present invention further relates to a stent made of a biodegradable polymer and to be deployed in a biliary duct. The stent including a first end and a second end defining a length therebetween. The stent further includes a plurality of first monofilaments and a plurality of second monofilaments braided at a first predefined braiding angle from the first end to the second end. The plurality of first monofilaments and the plurality of the second monofilaments are back braided at a second pre-defined braiding angle from the second end to the first end thus making a pair of the first monofilaments and a pair of the second monofilaments. The pair of first monofilaments and the pair of second monofilaments intersect with each other to define a pre-defined braiding pattern. Post axial compression, the length of the stent is decreased by 25% to 10% and the first and second pre-defined braiding angles of the stent are increased by 25% to 6.25%.

Furthermore, the present invention relates to a stent made of a biodegradable polymer and to be deployed in a biliary duct. The stent including a first end and a second end defining a length therebetween. The stent further includes a plurality of first monofilaments and a plurality of second monofilaments braided at a first predefined braiding angle from the first end to the second end, at least one of the plurality of first monofilaments or the plurality of the second monofilaments is back braided at a second pre-defined braiding angle from the second end to the first end. Post axial compression, the length of the stent is decreased by 25% to 10% and the first and second pre-defined braiding angles of the stent are increased by 25% to 6.25%.

The foregoing features and other features as well as the advantages of the invention will become more apparent from the following detailed description, which proceeds with reference to the accompanying figures.

### BRIEF DESCRIPTION OF ACCOMPANYING DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts an expanded view of a stent 100 in accordance with an embodiment of the present invention.

FIG. 1a depicts an expanded view of a stent 300 in accordance with an embodiment of the present invention.

FIG. 1b depicts a partial expanded view of a stent 400 in accordance with an embodiment of the present invention.

FIG. 1c depicts a partial expanded view of a stent 500 in accordance with an embodiment of the present invention.

FIG. 1d depicts the stent 300 after axial compression in accordance with an embodiment of the present invention.

FIG. 2 depicts a flow chart of a process 200 involved in manufacturing of the stent 100 in accordance with an embodiment of the present invention.

### DETAILED DESCRIPTION OF ACCOMPANYING DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although the operations of exemplary embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show the various ways in which the disclosed system, method, and apparatus can be used in combination with other systems, methods, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and appended claims, or may be learned by the practice of embodiments as set forth hereinafter. It should be noted that the term bio-resorbable, biodegradable or bio-absorbable are having same meaning and used either one in this submission.

In accordance with the present disclosure, a stent (or scaffold) and a method of manufacturing the stent are disclosed. The stent is a self-expanding stent. The stent is made of a biodegradable material. The stent can be implanted in narrow arteries of the digestive tract, respiratory tract, etc. In an embodiment, the stent is implanted in a biliary duct of the digestive tract of a patient. The stent can be absorbed in the body within a time period of six to eight months, thus avoiding elastic recoil of the stent and preventing various long-term complications such as inflammation and/or blockage, etc.

The stent of the present invention includes at least two sets of plurality of monofilaments, say a plurality of first monofilaments and a plurality of second monofilaments. The stent is manufactured by a process of braiding the at least two sets of plurality of monofilaments at a first predefined braiding angle to form a braided structure. Thereafter, at least one of the two sets of plurality of monofilaments is subjected to a step of back braiding of the loose ends of the said at least one of the two sets of plurality of monofilaments at a second predefined braiding angle. The back braiding process helps to maintain the shape and/or radial strength of the stent. In addition to the above, the back braiding forms closed ends of the stent. The closed ends of the stent are smooth therefore, prevents injury to the vessel during deployment.

Following the back braiding process, the stent is axially compressed at both ends. The process of axial compression improves shape memory and/or radial strength of the stent leading to enhanced shape recovery of the stent.

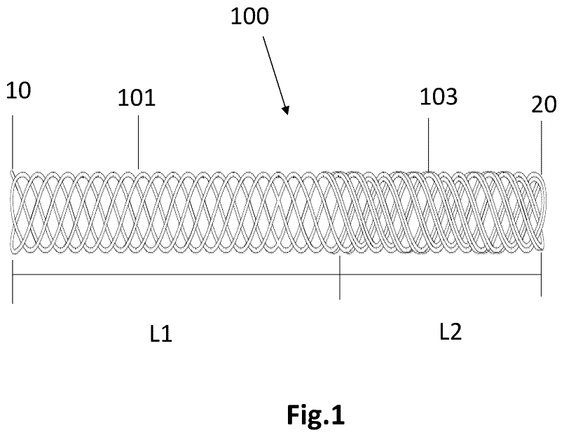

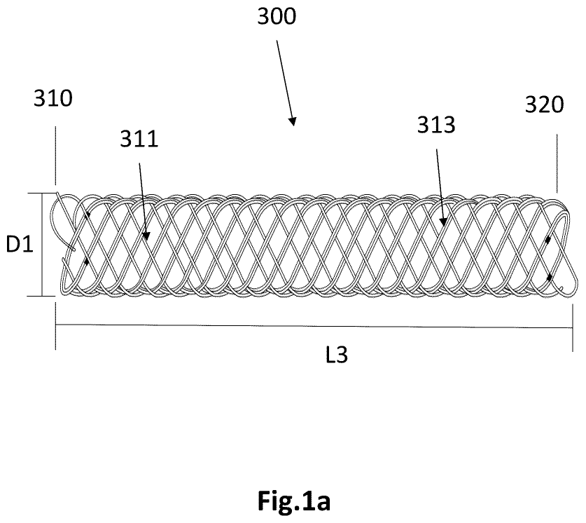

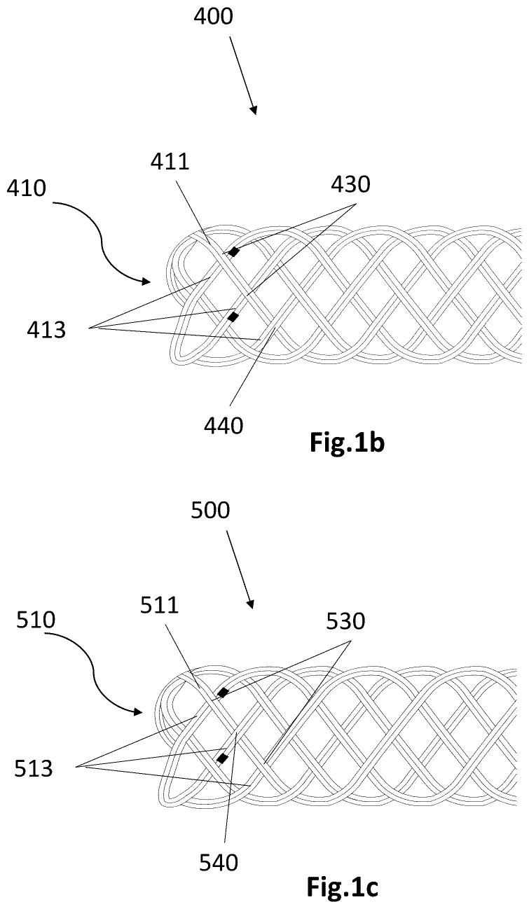

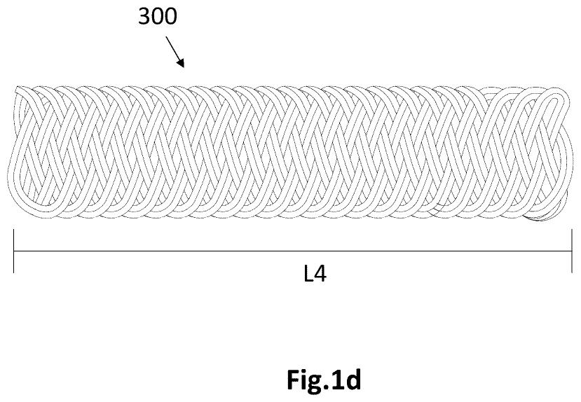

Now moving specifically to drawings, FIG. 1 represents an expanded view of a stent 100. The stent 100 may include a first end 10, a second end 20, and a length ‘L’ extending between the first end 10 and the second end 20. The length ‘L’ of the stent 100 may play a crucial role in determining the performance of the stent 100 in the native organ, for example, the biliary duct. The greater length of the stent 100 enables a greater area of the biliary duct to be lesion-free and/or provide a more uniform passage in the biliary duct after degradation of the stent 100. The length ‘L’ may be in the range of 40 mm to 100 mm. In an embodiment, the length ‘L’ is 50 mm.

The stent 100 may have a predefined diameter ‘D’. The diameter ‘D’ of the stent 100 plays a crucial in mitigating the risk of stent 100 migration while eliminating blockage and/or helps to impart required strength to the stent 100 after implantation at the treatment site. The diameter ‘D’ of the stent 100 may depend upon the location of the implantation. The diameter ‘D’ may be in the range of 8 mm to 10 mm. In an embodiment, the diameter ‘D’ is 8 mm.

The stent 100 may be a braided structure. The stent 100 may include a plurality of monofilaments braided together to form the braided structure. The number of a plurality of monofilaments may be in a range of 8 to 16. In an embodiment, the number of monofilaments is 12. The monofilaments may have a predefined diameter ranging from 0.300 mm to 0.500 mm. In an embodiment, the monofilaments have a diameter 0.400 mm. The lower diameter of the monofilaments results in a lower crimped profile of the stent 100 while maintaining desired strength of the stent 100 required for biliary stenting. The crimped profile of the stent 100 may be in a range of 3.3 mm to 4.2 mm and length 70 mm to 240 mm. In an embodiment, the crimped profile of the stent 100 is 3.7 mm and length 80 mm. The stent 100 may be deployed through a delivery sheath of diameter ranging from 11F to 14F. In an embodiment, the diameter of the delivery sheath is 12F.

The stent 100 may include at least two segments, for example, a first segment 101 towards the first end 10 and a second segment 103 towards the second end 20. The two segments may have different braiding densities to impart the required strength, stiffness, and flexibility to the stent 100.

In an embodiment, the second segment 103 may be densely braided as compared to the first segment 101. The second segment 103 may help in providing enhanced radial strength and/or stiffness to the stent 100. In addition, the second segment 103 help in better shape recovery of the stent 100.

The first segment 101 may include a length ‘L1’ in a range of 50% to 70% of the length L of the stent 100. In an embodiment, the length of the first segment 101 is 60% of the length ‘L’. The second segment 103 may include a length ‘L2’ in a range of 30% to 50% of the length L of the stent 100. In an embodiment, the length of the second segment 103 is 30% of the length ‘L’.

The first segment 101 and the second segment 103 may have same and/or different braiding angles. In an embodiment, the first segment 101 and the second segment 103 have same braiding angle. The first segment 101 and the second segment 103 may be braided at a predefined braiding angle ranging from 130° to 155°. In an embodiment, the predefined angle is 155°. The aforesaid braiding angle imparts enhanced radial strength to the stent 100.

In an embodiment, the first end 10 and the second end 20 of the stent 100 are closed ends. The closed ends are smooth and/or avoid injury to the vessel during deployment. Moreover, the closed ends at the first end 10 and the second end 20 help in maintaining stress distribution on the stent 100 resulting in uniform expansion of the stent 100 post deployment without affecting the strength of the stent 100.

The stent 100 may be provided with a plurality of markers (not shown) for enabling radiopacity of the stent 100. The markers may be attached on the first end 10 and the second end 20 of the stent 100. The markers may have a predefined shape but not limited to sleeve, circular etc. In an embodiment, the markers are in the shape of a sleeve.

The stent 100 may be made of biodegradable polymer. The biodegradable polymer may be selected from a group consisting of poly-L-lactide-co-caprolactone (PLC), poly dioxanone, poly ethylene glycol (PEG), polycaprolactone (PCL), poly-DL-lactic acid (PDLLA), poly L-lactide (PLLA), poly (glycolic acid) (PGA), poly L-lactide-co-glycolic acid (PLGA), and/or a combination thereof. In an embodiment, the stent 100 is made of polydioxanone. The polydioxanone has good shape retention and a rapid degradation rate of around six to eight months.

FIG. 1a depicts another embodiment of a stent 300. Similar to stent 100, the stent 300 includes a first end 310, a second end 320 and a length ‘L3’ extending between the first end 310 and the second end 320. In other words, the second end 320 defines the length ‘L3’ from the first end 310. The second end 320 may be disposed axially opposite to the first end 310. The length ‘L3’ may be in the range of 30 mm to 120 mm. In an embodiment, the length ‘L3’ is 80 mm.

The stent 300 may have a predefined diameter ‘D1’. The predefined diameter ‘D1’ of the stent 300 may depend upon the location of the implantation. The diameter ‘D1’ may be in the range of 6 mm to 12 mm. In an embodiment, the diameter ‘D1’ is 10 mm.

The crimped profile of the stent 300 may include a diameter ranging from 3.3 mm to 4.2 mm and a length ranging from 70 mm to 240 mm. In an embodiment, the diameter and length of the crimped profile of the stent 300 is 3.7 mm and 80 mm respectively. The stent 300 may be deployed through a delivery sheath of diameter ranging from 11 Fr to 14 Fr. In an embodiment, the diameter of the delivery sheath is 12 Fr.

Similar to the stent 100, the stent 300 may be a braided structure made of biodegradable polymer. The stent 300 may include at least two sets of a plurality of monofilaments, for example, a plurality of first monofilaments 311 and a plurality of second monofilaments 313. The plurality of monofilaments may have a predefined diameter ranging from 0.300 mm to 0.500 mm. In an embodiment, the monofilaments have a diameter 0.450 mm. The number of first monofilaments 311 and second monofilaments 313 may each range from 3 to 12 monofilaments. The number of first monofilaments 311 may be equal to or different than the number of second monofilaments 313. In an embodiment, the number of first monofilaments 311 and second monofilaments 313 are 6 and 3 respectively. The first monofilaments 311 and the second monofilaments 313 of the stent 300 may be selected from the monofilaments used to braid the stent 100 (described above).

At any lateral cross-section of the stent 300 across its length ‘L3’, the stent 300 may include a predefined ratio between the first monofilaments 311 and the second monofilaments 313. In an exemplary embodiment, as shown in FIG. 1a, the stent 300 includes two second monofilaments 313 for each first monofilament 311 across the length ‘L3’ of the stent 300.

In other words, at the time of braiding, the first monofilaments 311 are braided from the first end 310 to the second end 320. The second monofilaments 313 are braided from the first end 310 to the second end 320 and then the second monofilaments 313 are back-braided from the second end 320 to the first end 310. Thus, the stent 300 includes a pre-defined ratio between braided and back braided (first and second) monofilaments 311,313. In an exemplary embodiment, the pre-defined ratio between the braided and back braided monofilaments of stent 300 is 2:1.

The stent 300 includes high radial strength and high kink resistance due to the said pre-defined ratio between the first monofilaments 311 and the second monofilaments 313 which further enhances the holding capacity of the stent 300 to avoid migration. The stent 300 is quick to self-expand at the time of implantation. Furthermore, the stent 300 provides enhanced torque-ability and flexibility thereby easing the implantation of the stent 300.

The shape recovery of the stent 300 may be in the range of 70% to 100%. In an embodiment, the shape recovery of the stent 300 is 95%. The change in length and diameter of the stent 300 after expansion may range from 1% to 20%. In an embodiment, the change in length and diameter of the stent 300 is 5%. The enhanced shape recovery is an indicator of the improved radial strength of the stent 300. The radial strength of the stent 300 may be more than 20N.

Similar to stent 100, the stent 300 may be provided with a plurality of markers (not shown) for enabling radiopacity of the stent 300. The markers may be attached on the first end 310 and the second end 320 of the stent 300. The markers may have a predefined shape but not limited to sleeve, circular etc. In an embodiment, six sleeve shaped markers are provided over the stent 300.

FIG. 1b depicts yet another embodiment of a stent 400. The stent 400 is similar to the stent 300 except for the predefined ratio between a plurality of first monofilaments 411 and a plurality of second monofilaments 413. In this embodiment, the number of first monofilaments 411 and second monofilaments 413 are same, namely, 6 and 6 respectively.

At the time of braiding, the first monofilaments 411 and the second monofilaments 413 are braided from a first end 410 to the second end (not shown). And then, the first monofilaments 411 and the second monofilaments 413 are back-braided from the second end to the first end 410. Thus, the stent 400 includes a pre-defined ratio between braided and back braided (first and second) monofilaments 411,413. In an exemplary embodiment, the pre-defined ratio between the braided and back braided monofilaments of the stent 400 is 1:1. Due to back braiding of both the first monofilaments 411 and the second monofilaments 413, the stent 400 exhibits very high radial strength and quick shape recovery after deployment.

The shape recovery of the stent 400 may be in the range of 80% to 100%. In an embodiment, the shape recovery of the stent 400 is 95%. The change in length and diameter of the stent 400 after expansion may range from 1% to 15%. In an embodiment, the change in length and diameter of the stent 400 is 5%. The enhanced shape recovery is an indicator of the improved radial strength of the stent 400. The radial strength of the stent 400 may be more than 20N.

The first monofilaments 411 and the second monofilaments 413 of the stent 400 may be braided in a pre-defined braiding pattern including a plurality of first nodes 430 and a plurality of second nodes 440. Each of the first nodes 430 and the second nodes 440 may include one crossing (or intersection) of a pair of first monofilaments 411 and a pair of second monofilaments 413.

The pair of first monofilaments 411 braided over the pair of second monofilaments 413 intersects at the first node 430. Similarly, the pair of first monofilaments 411 braided under the pair of second monofilaments 413 intersects at the second node 440.

In an exemplary embodiment, as shown in FIG. 1b, the braiding pattern of the stent 400 includes the pair of first monofilaments 411 intersects with the pair of second monofilaments 413 at two first nodes 430 for every intersection at the second node 440.

FIG. 1c depicts yet another embodiment of a stent 500. The stent 500 is similar to the stent 400 except for the braiding pattern. In an exemplary embodiment, as shown in FIG. 1c, the braiding pattern of the stent 500 includes a pair of first monofilaments 511 alternately intersecting at a first node 530 and a second node 540 with a pair of second monofilament 513. The number of first monofilaments 511 may be equal to or different than the number of second monofilaments 513. In an embodiment, the number of first monofilaments 511 and second monofilaments 513 are 6 and 6 respectively. The braiding pattern of the stent 500 imparts excellent radial strength to the stent 500 with quick shape recovery.

The shape recovery of the stent 500 may be in the range of 80% to 100%. In an embodiment, the shape recovery of the stent 500 is 95%. The change in length and diameter of the stent 500 after expansion may range from 1% to 15%. In an embodiment, the change in length and diameter of the stent 500 is 5%. The enhanced shape recovery is an indicator of the improved radial strength of the stent 500. The radial strength of the stent 500 may be more than 20N.

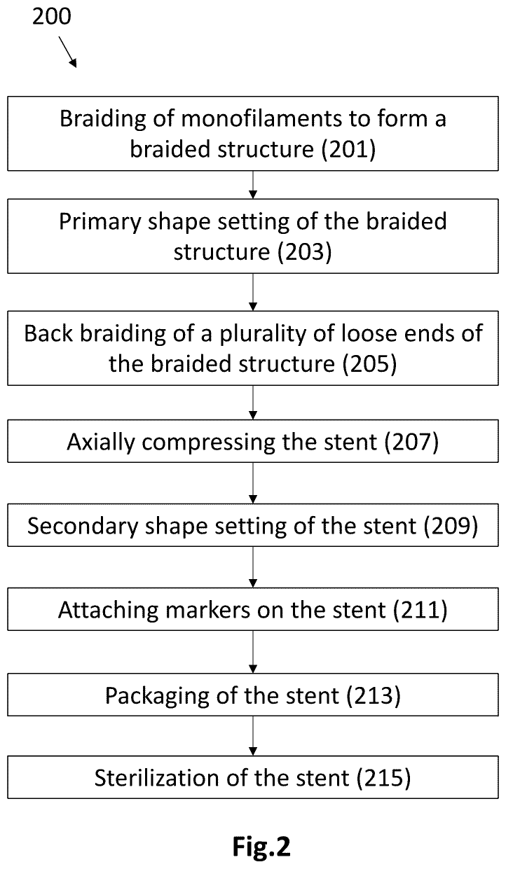

In accordance with an embodiment of the present invention, FIG. 2 depicts a process (or method) 200 involved in the manufacturing of the stent 300. The process of manufacturing commences by braiding the plurality first monofilaments 311 and the plurality of second monofilaments 313 from the first end (310) to the second end (320) at a first predefined braiding angle to form a braided structure at step 201. The first predefined braiding angle may be in a range of 120° to 160°. In an embodiment, the first predefined braiding angle is 145°. The number of first monofilaments 311 and second monofilaments 313 may each range from 3 to 12 monofilaments. In an embodiment, the number of first monofilaments 311 and second monofilaments 313 are 6 and 3 respectively. The braided structure having the length ‘L3’.

The first monofilaments 311 and the second monofilaments 313 may be attached to a mandrel (not shown), including a plurality of hooks. The plurality of first monofilaments 311 and the second monofilaments 313 may be grooved on the plurality of hooks. The mandrel may have a predefined diameter ranging from 6 mm to 12 mm. In an embodiment, the diameter of the mandrel is 10 mm. The braiding of the plurality of first and second monofilaments 311, 313 may be performed using an automatic braiding machine (not shown) to form a braided structure (not shown). The braiding machine may include a plurality of carriers. In an embodiment, the braiding machine includes 24 carriers. The carriers may be present in 12 sets of two carriers. The process of braiding may be performed by wrapping each portion of the first monofilaments 311 and the second monofilaments 313 in spools of one set of the carriers in an alternate manner.

Post braiding at step 201, the braided structure is subjected to a process of primary shape setting at step 203. The primary shape setting may be performed to obtain the desired shape. The process of primary shape setting may be performed by heating the braided structure in an oven at a predefined temperature for a predefined time duration. The predefined temperature and predefined time duration may be in a range of 80° C. to 100° C. and 14 hours to 16 hours, respectively. In an embodiment, the process of primary shape setting is performed at a temperature of 90° C. for 15 hours.

After the process of primary shape setting, a plurality of loose ends (of at least one of the first monofilaments 311 and/or second monofilaments 313) of the braided structure is subjected to a process of back braiding from the second end 320 to the first end 310 at a second predefined braiding angle to form the stent 300 at step 205. In an exemplary embodiment, the plurality of the second monofilaments is subjected to the process of back braiding to form the stent 300. The second predefined braiding angle may be in a range of 120° to 160°. In an embodiment, the second predefined angle is 145°. The process of back braiding may be performed by loading the braided structure on the mandrel in a reverse direction. All the loose ends of braided structure may be cut to a predefined length ranging from 30 mm to 120 mm. In an embodiment, the predefined length is 55 mm. The process of back braiding may be performed manually. The back braiding process helps to maintain the shape and/or radial strength of the stent 300. In addition to the above, the back braiding forms closed ends of the stent 300. The closed ends of the stent 300 are smooth therefore, prevents injury to the vessel during deployment. The pre-defined ratio between the braided and back braided monofilaments of stent 300 is 2:1.

Post back braiding, at step 207, the stent 300 is axially compressed by applying a pre-defined force to from the compressed stent. The pre-defined force ranges from 0.015N to 0.02N. In an exemplary embodiment, the process of axial compression may be performed at both the ends 310, 320 of the stent 300 by manually applying a force of 0.018N. The stent 300 may be axially compressed to decrease the length ‘L3’ (as shown in FIG. 1b) to a predefined length ‘L4’ (as shown in FIG. 1d). The length ‘L4’ may be in a range of 25% to 10% of the length ‘L3’ of the stent 300. In an embodiment, the length ‘L4’ is reduced to 20% from the length ‘L3’. The process of axial compression may be performed by means of compression using a mandrel with a fixture. The fixture of the mandrel may be pressed manually, leading to the desired compression of the stent 300.

The process of axial compression of the stent 300 increases the first and second pre-defined braiding angle and decreases the length ‘L3’ of the stent 300. The braiding angle and length after the compression may range from 150° to 170° and 20 mm to 110 mm respectively. In an embodiment, the braiding angle is 155° and the length is 40 mm. Thus, the first and second pre-defined braiding angle is increased by a third pre-defined angle that is 25% to 6.25% of the first and second pre-defined angle. The increased braiding angle enhances the radial strength of the stent 300. The process of axial compression enhances the strength and/or recoil capacity of the stent 300. Moreover, axial compression renders the stent 300 with better shape memory and/or retention capacity. Therefore, the stent 300 has minimal changes in the length and diameter after it is expanded from the compressed state at the implantation site.

Following axial compression, the compressed stent 300 is subjected to a process of secondary shape setting at step 209. The process of secondary shape setting may be performed in a vacuum heating oven at the predefined temperature ranging from 80° C. to 100° C. for the predefined time duration of 14 hours to 16 hours. In an embodiment, the process of secondary shape setting is performed at a temperature of 90° C. for 15 hours. A pressure ranging from between 600 mmHg to 700 mmHg may be generated by vacuum to remove internal stress from the first and second monofilaments 311, 313.

The process of secondary shape setting enhances the longitudinal and radial strength of the stent 300 and/or prevents changes in the braiding angle of the first and second monofilaments 311, 313 after expansion. The longitudinal and radial strength of the stent 300 may be more than 20N. In an embodiment, the strength of the stent 300 is 20N. Post-secondary shape setting, the braided structure is allowed to cool gradually till it attains 25° C. to 30° C. temperature over a period of duration. Additionally, post-secondary shape setting, a plurality of loose ends of the plurality of first and second monofilaments 311, 313 are manually cut and attached to adjacent first and second monofilaments 311, 313. The loose ends of the plurality of first and second monofilaments 311, 313 are attached together by means of welding, adhesives, etc. In an embodiment, the loose ends of the plurality of first and second monofilaments 311, 313 is attached by means of adhesives.

Post cutting, radiopaque markers are attached at both ends 310, 320 of the stent 300 at step 211. The markers may be attached by means of without limitation, crimping, bonding, welding, adhesive, etc. In an embodiment, the markers are attached by means of crimping.

The stent 300 is subjected to a process of packaging at step 213. The process of packaging may be performed by placing the stent 300 in an aluminum pouch. After placement, the pouch is vacuum-sealed and packed inside a box at room temperature.

Post packaging, the stent 300 is subjected to a process of sterilization 215. The sterilization may be performed using a radiation sterilization process, such as without limitation e-beam radiation sterilization, Ethylene oxide gas sterilization or gamma sterilization. In an embodiment, the coated implant is sterilized using gamma radiation sterilization. The sterilization is done at a dose of gamma radiation in a range of 20 kGy to 30 kGy, more preferably 23 kGy to 27 kGy.

One or more steps of the process 200 may be modified to manufacture stent 400, 500. In an exemplary embodiment, the step 205 is modified to make the stent 400, 500. The plurality of the first monofilaments 411, 511 and the plurality of the second monofilaments 413, 513 are subjected to the process of back braiding to form the stent 400, 500. The process of back braiding yields the pair of the first monofilaments 411, 511 and the pair of the second monofilaments 413, 513. For the stent 400 (as shown in FIG. 1b) while back braiding, the pair of first monofilament 411 intersects with the pair of second monofilament 413 at two first nodes 430 for every intersection at a second node 440. For the stent 500 (as shown in FIG. 1c) while back braiding, the pair of first monofilament 511 alternately intersects at one first node 530 and one second node 540 with the pair of second monofilament 513. The pre-defined ratio between the braided and back braided (first and second) monofilaments 411, 511, 413, 513 of the stent 400, 500 is 1:1.

The invention will now be described with the help of the following examples.
Example 1 (Present Invention): Polydioxanone (PDO) monofilaments having diameter of 450 micron was used to make the braided structure. The six first monofilaments 311 and three second monofilaments 313 were loaded into 24 carrier automatic braiding machine. There were a total 12 sets of the carrier with each set containing two carriers. The six first monofilaments 311 and three second monofilaments 313 were grooved in respective hook pins of a mandrel. Thereafter, the first and second monofilaments 311, 313 were braided automatically to form a braided structure of length 50 mm at a braiding angle of 145°. Post braiding, the braided structure was subjected to a process of primary shape setting. The process of primary shape setting was performed at a temperature of 90° C. for 15 hours. After the process of primary shape setting, the first monofilaments 311 of the braided structure was back braided at a braiding angle of 145°. After back braiding, the stent 300 was axially compressed to a length of 40 mm. The compressed stent 300 was then subjected to a process of secondary shape setting at a temperature of 90° C. for 15 hours inside a vacuum heating oven.

The stent 300 was then loaded in a delivery sheath in a crimped state and subjected to expansion at the implantation site. It was found that the length of the stent 100, after expansion, increased from 40 mm to 44 mm which was less than 10% increase compared to original length. The braiding angle also increased from to 145° to 155°. Therefore, it was observed that the stent 300 recovered 90% of its shape which is an indicator of enhanced radial strength. The radial strength of the stent 300 was measured and found to be more than 20N. Therefore, the stent 300 was able to restore the patency of the biliary duct.
Example 2 (Prior art): The commercially available polydioxanone (PDO) monofilaments having diameter of 450 micron was used to make the braided tubular structure. The six monofilaments were loaded on to 24 carrier automatic braiding machine. There was total 12 sets of the carrier with each set containing two carriers. The mandrel had six hook pins at one end. The six monofilaments were grooved in the hook pins. Following, the monofilaments were braided automatically to make a braided structure of length 50 mm at a braiding angle of 145°. Post braiding, the braided structure was subjected to a process of primary shape setting. The process of primary shape setting was performed at a temperature of 90° C. for 15 hours. Further, the braided tubular structure was back braided and again shape set at the temperature of the 90° C. for 15 hours. After that stent was loaded in a delivery sheath in crimped state and was subjected to expansion at the implantation site.

After expansion, it was observed that the stent did not recover its shape. The length and diameter of the stent were measured using vernier caliper. The length of the stent was found to be increased from 50 mm to 63 mm and the diameter was found to be decreased to 6.3 mm. The change in length and diameter of the stent was more than 20% than its original length and diameter. Further, the braiding angle of the stent was found to be reduced to 135°. The stent was tested for measuring the radial strength which was found to 8N which is less than the required range. The stent was not able to restore the patency of the biliary duct.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. A method (200) of manufacturing a stent (300, 400, 500) from a biodegradable polymer, the method (200) comprising:
braiding at least a plurality of first monofilaments (311, 411, 511) and a plurality of second monofilaments (313, 413, 513) from a first end (310) to a second end (320) at a first predefined braiding angle to form a braided structure, the braided structure having a length (L3) between the first end (310) and the second end (320), the second end (320) being axially opposite to the first end (310);
primary shape setting of the braided structure at a predefined temperature for a predefined time duration;
back braiding at least one of the first monofilaments (311, 411, 511) or the second monofilaments (313, 413, 513) from the second end (320) to the first end (310) at a second pre-defined braiding angle to form the stent (300, 400, 500);
axially compressing the stent (300, 400, 500) by applying a pre-defined force to decrease the length (L3) by 25% to 10% of the length (L3) of the stent (300, 400, 500), and increase the first and second predefined braiding angle of the stent (300, 400, 500) by 25% to 6.25% of the first and second pre-defined angle of the stent (300, 400, 500); and
secondary shape setting the stent (300, 400, 500) at the predefined temperature for the predefined time period.

2. The method (200) as claimed in claim 1, wherein the first predefined braiding angle and the second predefined angle ranges from 120° to 160°.

3. The method (200) as claimed in claim 1, wherein the pre-defined force ranges from 0.015N to 0.02N.

4. The method (200) as claimed in claim 1, wherein the step of primary shape setting includes heating the braided structure at the predefined temperature ranging from 80° C. to 100° C. for the predefined time duration ranging from 14 hours to 16 hours.

5. The method (200) as claimed in claim 1, wherein the step of secondary shape setting includes heating the stent (300, 400, 500) at a pressure ranging from 600 mmHg to 700 mmHg generated by vacuum and at the predefined temperature ranging from 80° C. to 100° C. for the predefined time duration ranging from 14 hours to 16 hours.

6. The method (200) as claimed in claim 1, wherein the step of back braiding includes back braiding at least one of the first monofilaments (311, 411, 511) or the second monofilaments (313, 413, 513) in a pre-defined ratio between braided and back-braided first monofilaments (311, 411, 511) and second monofilaments (313, 413, 513) of either 2:1 or 1:1.

7. A stent (400, 500) made of a biodegradable polymer and to be deployed in a biliary duct, the stent (400, 500) comprising:
a first end;
a second end defining a length (L3) of the stent (400, 500) from the first end;
a plurality of first monofilaments (411, 511) and a plurality of second monofilaments (413, 513) braided at a first predefined braiding angle from the first end to the second end, the plurality of first monofilaments (411, 511) and the plurality of the second monofilaments (413, 513) are back braided at a second pre-defined braiding angle from the second end to the first end thus making a pair of the first monofilaments (411, 511) and a pair of the second monofilaments (413, 513), the pair of first monofilaments (411, 511) and the pair of second monofilaments (413, 513) intersect with each other to define a pre-defined braiding pattern;
wherein, post axial compression, the length (L3) of the stent (400, 500) is decreased by 25% to 10% and the first and second pre-defined braiding angles of the stent (400, 500) are increased by 25% to 6.25%.

8. The stent (400) as claimed in claim 7, wherein the pair of first monofilaments (411) intersects with the pair of second monofilaments (413) at two first nodes (430) for every intersection at a second node (440).

9. The stent (500) as claimed in claim 7, wherein the pair of first monofilaments (511) alternately intersects at a first node (530) and a second node (540) with the pair of second monofilaments (513).

10. The stent (400, 500) as claimed in claim 7, wherein the first and second pre-defined braiding angle ranges from 120° to 160°.

11. The stent (400, 500) as claimed in claim 7, wherein a pre-defined ratio between braided and back-braided first monofilaments (411, 511) and second monofilaments (413, 513) is 1:1.

12. The stent (400, 500) as claimed in claim 7, wherein a longitudinal and radial strength of the stent (400, 500) is more than 20N.

13. A stent (300) made of a biodegradable polymer and to be deployed in a biliary duct, the stent (300) comprising:
a first end (310);
a second end (320) defining a length (L3) of the stent (300) from the first end (310);
a plurality of first monofilaments (311) and a plurality of second monofilaments (313) braided at a first predefined braiding angle from the first end (310) to the second end (320), at least one of the plurality of first monofilaments (311) or the plurality of the second monofilaments (313) is back braided at a second pre-defined braiding angle from the second end (320) to the first end (310);
wherein, post axial compression, the length (L3) of the stent (300) is decreased by 25% to 10% and the first and second pre-defined braiding angles of the stent (300) are increased by 25% to 6.25%.

14. The stent (300, 400, 500) as claimed in claim 14, wherein a pre-defined ratio between braided and back-braided first monofilaments (311) and second monofilaments (313) is 2:1.
